(19) United States
(12) Patent Application Publication
(10) Pub. No.: US 2021/0307989 A1
(43) Pub. Date: Oct. 7, 2021

(54) Treatment Positioning Headrest

(71) Applicant: Frank Fadeley, Hamburg, NY (US)

(72) Inventor: Frank Fadeley, Hamburg, NY (US)

(21) Appl. No.: 17/222,272

(22) Filed: Apr. 5, 2021

Related U.S. Application Data
(60) Provisional application No. 63/005,645, filed on Apr. 6, 2020.

(51) Int. Cl. A61G 13/12

(52) U.S. Cl. CPC A61G 13/121

(57) ABSTRACT

A treatment positioning headrest is provided. The device includes a headrest base having a front side, a rear side, and a pair of lateral sides extending about a perimeter of an upper side of the headrest base. A planar flange extends from a distal end of each of the front side, rear side, and the pair of lateral sides. A depression is defined in the upper side, wherein the depression is dimensioned to conform to a rear portion of a head. A longitudinal ruler is disposed on each lateral side, wherein each longitudinal ruler comprises a plurality of measurement indicia therealong. In some embodiments, a pair of extensions rise from the rear side of the headrest base, wherein the pair of extensions retain the neck therebetween.

### CROSS REFERENCE TO RELATED APPLICATIONS

This application claims the benefit of U.S. Provisional Application No. 63/005,645 filed on Apr. 6, 2020. The above identified patent application is herein incorporated by reference in its entirety to provide continuity of disclosure.

### BACKGROUND OF THE INVENTION

The present invention relates to medical treatment headrests. More particularly, the present invention pertains to a headrest securable to a medical treatment table for positioning the head of a patient at a precise location.

Many individuals suffer from cancer or other illnesses that require an accurately delivered treatment, such as radiation therapy, to the affected area. However, such treatments can be difficult to properly administer, as the patient must be constantly repositioned to ensure that the treatment is properly directed at the desired area, Additionally, subtle head tilts can be difficult for medical professionals to identify during treatment, which can result in the treatment becoming ineffective. In addition to being ineffective, improperly directed treatment can expose otherwise healthy parts of the patient's body to harmful radiation. Therefore, a device that allows a user to precisely orient and align a patient's head for medical treatment is desired.

In light of the devices disclosed in the known art, it is submitted that the present invention substantially diverges in design elements from the known art and consequently it is clear that there is a need in the art for an improvement to existing medical treatment headrests. In this regard, the instant invention substantially fulfills these needs.

### SUMMARY OF THE INVENTION

In view of the foregoing disadvantages inherent in the known types of medical treatment headrests now present in the known art, the present invention provides a medical treatment headrest wherein the same can be utilized for providing convenience for the user when precisely positioning a patient's head during a medical treatment.

The present system comprises a headrest base having a front side, a rear side, and a pair of lateral sides extending about a perimeter of an upper side of the headrest base. A planar flange extends from a distal end of each of the front side, rear side, and the pair of lateral sides. A depression is defined in the upper side, wherein the depression is dimensioned to conform to a rear portion of a patient's head. A longitudinal ruler is disposed on each lateral side, wherein each longitudinal ruler comprises a plurality of measurement indicia therealong. In some embodiments, a pair of extensions rise from the rear side of the headrest base, wherein the pair of extensions are dimensioned to receive a neck of the patient therebetween.

In some embodiments, a height of the rear side is greater than a height of the front side. In another embodiment, the depression comprises an arcuate shape along an entire length between the front side and the rear side. In other embodiments, a plurality of apertures is disposed through the planar flange, wherein the plurality of apertures is configured to receive a fastener therethrough to secure the headrest to a treatment table. In yet another embodiment, the longitudinal rulers are etched into the pair of lateral sides. In some embodiments, reference indicia are disposed on each lateral side adjacent to the rear side, wherein the reference indicia are associated with a contour of a plurality of headrest bases. In another embodiment a depth of the depression defined in the upper side is less than a height of each of the front side, rear side, and the pair of lateral sides. In other embodiments, the longitudinal rulers are mirrored relative to each other. In some embodiments, an arcuate cutout is defined between the pair of extensions. In another embodiment, the depression extends through the front side such that an upper edge of the front side defines an arcuate shape.

### BRIEF DESCRIPTION OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of an embodiment of the treatment positioning headrest.

FIG. 2 shows a side view of the lateral side of an embodiment of the treatment positioning headrest.

FIG. 3A shows a perspective view of an alternate embodiment of the treatment positioning headrest.

FIG. 3B shows a perspective view of an alternate embodiment of the treatment positioning headrest.

FIG. 3C shows a perspective view of an alternate embodiment of the treatment positioning headrest.

FIG. 3D shows a perspective view of an alternate embodiment of the treatment positioning headrest.

FIG. 4 shows a perspective view of an embodiment of the treatment positioning headrest in use.

### DETAILED DESCRIPTION OF THE INVENTION

Reference is made herein to the attached drawings. Like reference numerals are used throughout the drawings to depict like or similar elements of the treatment positioning headrest. The figures are intended for representative purposes only and should not be considered to be limiting in any respect.

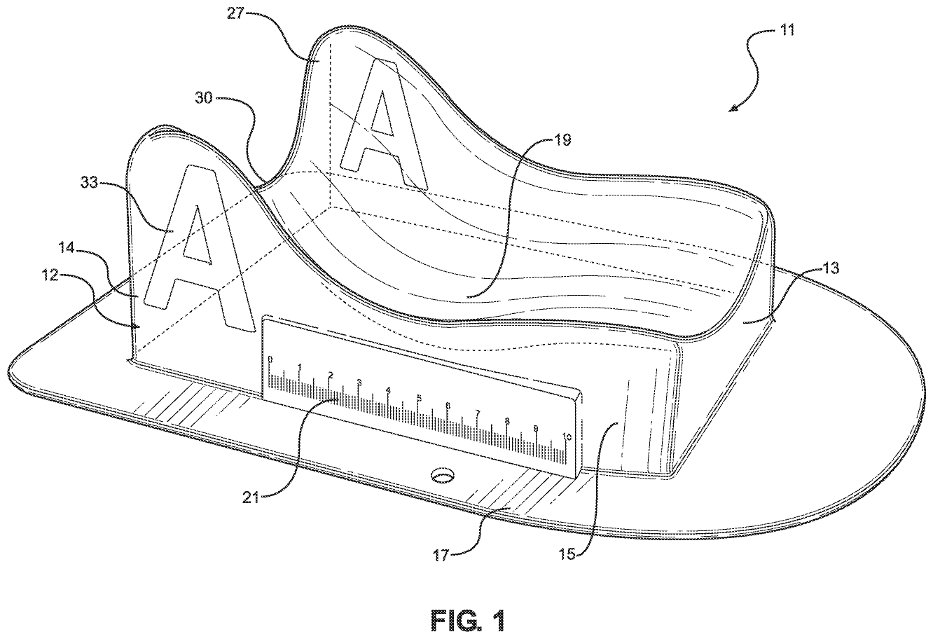

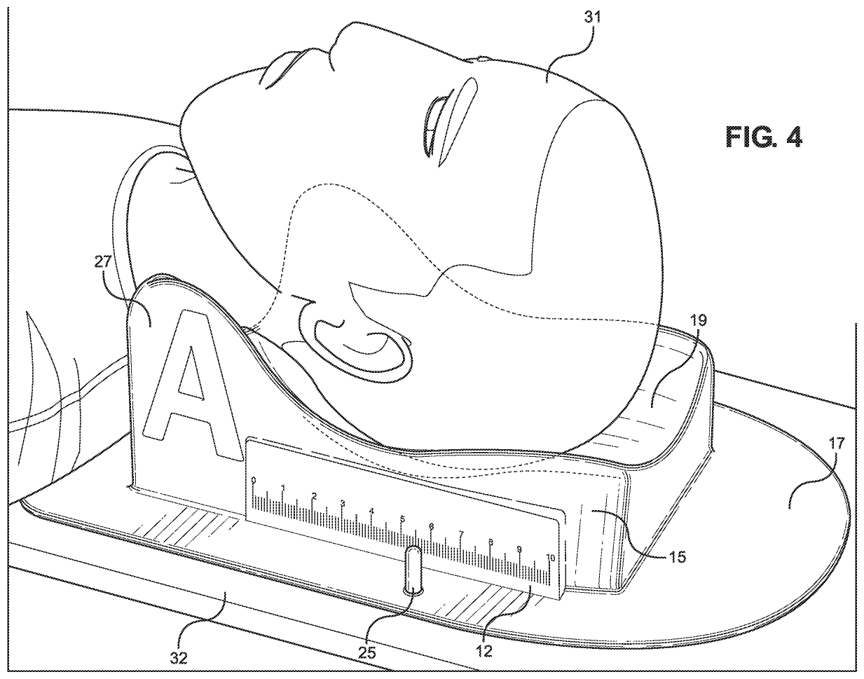

Referring now to FIG. 1, there is shown a perspective view of an embodiment of the treatment positioning headrest. The treatment positioning headrest 11 comprises a headrest base 12 having a front side 13, a rear side 14, and a pair of lateral sides 15 extending from an upper side of the headrest base 12. In operation, a patient's neck is disposed on the rear side 14 and an upper portion of the patient's head is disposed adjacent to the front side 13. In the illustrated embodiment, each side, 13, 14, 15 of the headrest base 12 extends substantially orthogonally from the upper side to define a raised headrest base 12. A depression 19 is defined in the upper side of the headrest base 12, wherein the depression 19 is dimensioned to receive a rear portion of a patient's head therein. In the shown embodiment, the depression 19 defines a rounded form factor configured to contour to an occipital portion of a patient's skull to retain the patient's head within the headrest base 12 for stability and comfort during medical treatments. In some embodiments, the depression 19 defines an arcuate cross-section across an entirety thereof, such that a depth of the depression 19 is greatest at a point furthest from the pair of lateral sides 15. In the illustrated embodiment, a planar flange 17 extends from a distal end of each side 13, 14, 15 of the headrest base 12, wherein the planar flange 17 provides structural stability to the headrest base 12 when secured to a support surface, such as a procedure table (as shown in FIG. 4, 32). In the illustrated embodiment, the planar flange 17 includes an arcuate front end and a linear rear end to conform to typical support platforms and procedure table headrests. A longitudinal ruler 21 is disposed along at least one of the pair of lateral sides 15, wherein the longitudinal ruler 21 provides a means of accurately and precisely aligning a patient's head along the headrest base 12 to position the patient for medical treatment. In the shown embodiment, the headrest base 12 further comprises reference indicia 33 disposed on each lateral side 15 of the headrest base, 12 wherein the reference indicia 33 correspond to variations of headrest base 12 geometry as further described elsewhere herein.

In the illustrated embodiment, a pair of extensions 27 extend from the headrest base 12 along the rear side 14, wherein the pair of extensions 27 serve to retain a patient's neck therebetween, such that the patient's head is disposed within the depression 19. In the shown embodiment, the rear side 14 comprises a height greater than the front side 13, such that the patient's neck is properly supported by the rear side 14. An arcuate cutout 30 is defined between the pair of extensions 27, wherein the arcuate cutout 30 is configured to provide a comfortable resting surface for a patient's neck. In some embodiments, the arcuate cutout 30 defines a rounded edge to prevent the arcuate cutout 30 from uncomfortably digging into a patient's neck. As such, a greater proportion of the patient's head and neck is in contact with the treatment positioning headrest 11 to increase frictional engagement with the patient to increase stability and ensure that the patient's head remains properly positioned throughout the medical treatment.

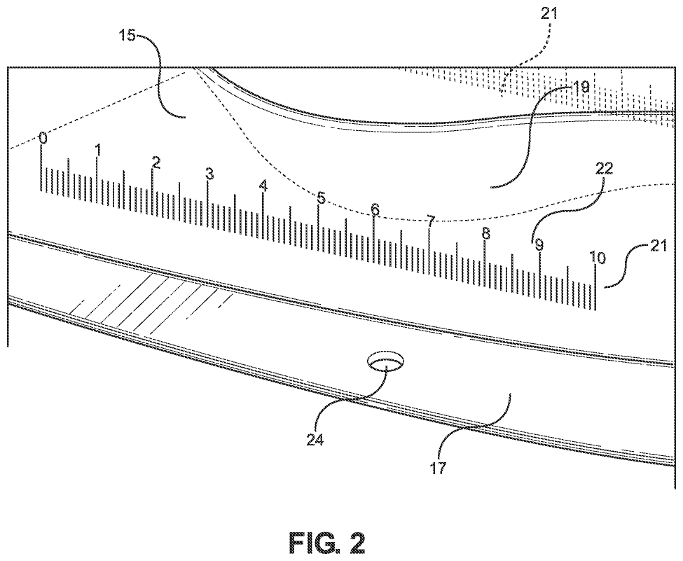

Referring now to FIG. 2, there is shown a side view of the lateral side of an embodiment of the treatment positioning headrest. The longitudinal ruler 21 extends along at least one lateral side of the pair of lateral sides 15, wherein the longitudinal ruler 21 comprises a plurality of measurement indicia 22 therealong. In the shown embodiment, the plurality of measurement indicia 22 comprise the numbers 0 through 10, however, in alternate embodiments, the plurality of measurement indicia 22 can include a variety of units and measurement subdivisions to provide additional precision in measuring the position of the patient's head within the depression 19. In some embodiments, each end of the longitudinal ruler 21 is aligned with an interior surface of each of the front side and the rear side of the headrest base, such that the patient's head can be positioned along an entirety of the headrest base. Similarly, in embodiments including the pair of extensions, one end of the longitudinal ruler 21 is aligned with an interior surface of the pair of extensions. In the illustrated embodiment, a longitudinal ruler 21 is disposed on each lateral side 15 of the headrest base, such that the patient's head can be properly positioned from opposite sides of the treatment positioning headrest. In some such embodiments, the longitudinal rulers 21 on each lateral side 15 are mirrored relative to each other. In other embodiments, the longitudinal rulers 21 are etched into the material of the pair of lateral sides 15 ensuring that the longitudinal rulers 21 are permanently disposed within the treatment positioning headrest and cannot be worn away after repeated use.

In the shown embodiment, an aperture 24 is disposed through the planar flange 17, wherein the aperture 24 is dimensioned to receive a fastener therethrough, such that the treatment positioning headrest can be secured to a procedure table. Additional apertures 24 can be disposed through the planar flange 17 to secure the headrest to the procedure table at multiple points. In the shown embodiment, a depth of the depression 19 is less than a height of each of the front side, rear side, and pair of lateral sides 15. As such, the depression 19 does not extend beyond the plane defined by the planar flange 17, allowing the treatment positioning headrest to rest flush on the procedure table during use.

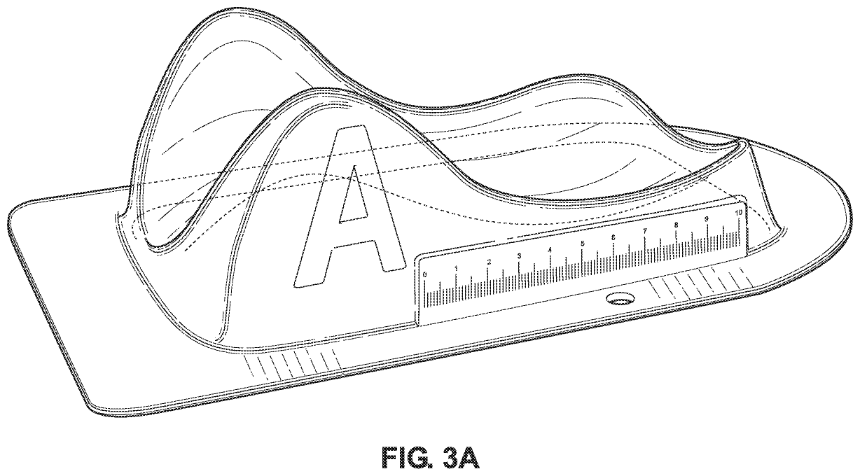

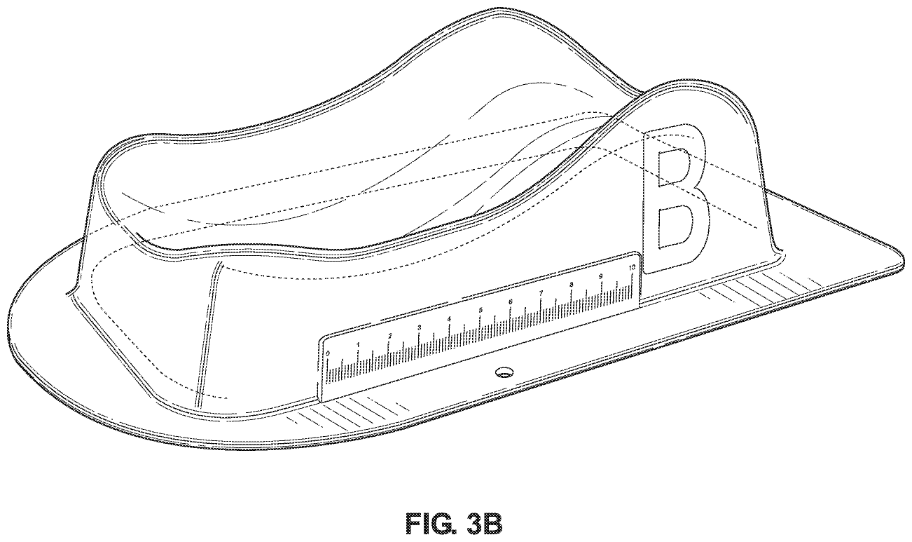

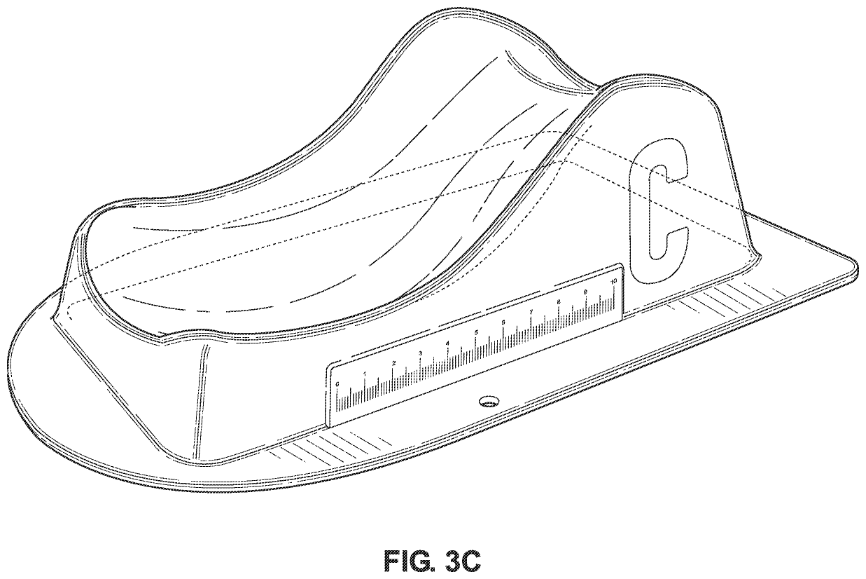

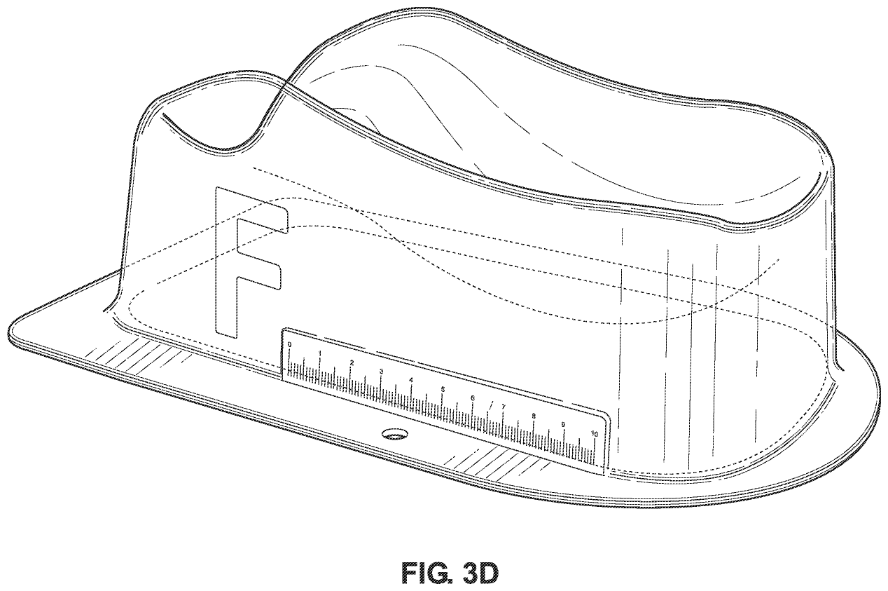

Referring now to FIGS. 3A, 33, 3C, and 3D, there are shown perspective views of various embodiments of the treatment positioning headrest. In the illustrated embodiments, a variety of differing formats for the treatment positioning headrest are provided, wherein each of the differing formats is beneficial to orient a patient's head for a particular targeted treatment. For example, in the illustrated embodiments of FIGS. 3A through 3C, the elevated rear side provides a depression in which a patient's head is in an inclined position, wherein the top of the patient's head is disposed lower relative to the patient's neck. The relative heights of the rear side and the front side can vary as needed to provide inclines of differing angles to ensure that the patient's head is positioned at a proper angle. Particularly, it should be noted that the depth of the arcuate cutout between the pair of extensions can similarly vary to alter the angle of the incline for the patient's head. Furthermore, in the embodiment of FIG. 3D, the relative heights of the front and rear sides are approximately equal, thereby providing a relatively flat disposition of the patient's head, ensuring that the user can properly position the patient's head for precise treatment via targeted therapy.

Referring now to FIG. 4, there is shown a perspective view of an embodiment of the treatment positioning headrest in use. In operation, the headrest is affixed to a procedure table 32 via a fastener 25 inserted through the aperture within the planar flange. In some embodiments, the fastener 25 comprises a securement peg permanently affixed to the procedure table 32, wherein the securement peg extends through the aperture to retain the headrest thereon. Once secured to the procedure table 32, the patient's head 31 can be placed within the depression 19 such that the user's neck is disposed in the arcuate edge of the rear side of the headrest base. The upper portion of the patient's head 31 can be placed adjacent to the front side, such that the patient's head 31 is retained within the depression 19. In the shown embodiment, the depression 19 further defines arcuate edges along an upper side of each lateral side 15 of the headrest base, such that the patient's ears are not contacted by the headrest base. Additionally, this forms a curved depression 19 extending between the front side and the rear side, wherein the depression 19 falls from the rear side towards a central lowest point, and then rises towards the front side at a height less than the front side. Once placed within the depression 19, the patient's head 31 can be more precisely aligned via the longitudinal ruler 21. In this manner, the user can precisely align a patient's head 31 for treatment. In some embodiments, once properly positioned, the patient's head 31 can be further secured on the headrest via medical restraints. In this manner, the patient's head 31 can be maintained in a desired location during an entirety of a precision medical treatment, such as targeted radiation.

It is therefore submitted that the instant invention has been shown and described in various embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly, and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

1. A treatment positioning headrest, comprising:
a headrest base having a front side, a rear side, and a pair of lateral sides extending about a perimeter of an upper side of the headrest base;
a planar flange extending from a distal end of each of the front side, rear side, and the pair of lateral sides;
wherein a depression is defined in the upper side, wherein the depression is dimensioned to conform to a rear portion of a head;
a longitudinal ruler disposed on each lateral side, wherein each longitudinal ruler comprises a plurality of measurement indicia therealong.

2. The treatment positioning headrest of claim 1, wherein a height of the rear side is greater than a height of the front side.

3. The treatment positioning headrest of claim 1, wherein the depression comprises an arcuate shape along an entire length between the front side and the rear side.

4. The treatment positioning headrest of claim 1, further comprising a plurality of apertures through the planar flange, the plurality of apertures configured to receive a fastener therethrough.

5. The treatment positioning headrest of claim 1, wherein the longitudinal rulers are etched into the pair of lateral sides.

6. The treatment positioning headrest of claim 1, further comprising reference indicia disposed on each lateral side adjacent to the rear side, wherein the reference indicia is associated with a contour of a plurality of headrest bases.

7. The treatment positioning headrest of claim 1, wherein a depth of the depression defined in the upper side is less than a height of the front side, the rear side, and the pair of lateral sides.

8. The treatment positioning headrest of claim 1, wherein the longitudinal rulers are mirrored relative to each other.

9. A treatment positioning headrest, comprising:
a headrest base having a front side, a rear side, and a pair of lateral sides extending about a perimeter of an upper side of the headrest base;
a planar flange extending from a distal end of each of the front side, rear side, and the pair of lateral sides;
wherein a depression is defined in the upper side, wherein the depression is dimensioned to conform to a rear portion of a head;
a longitudinal ruler disposed on each lateral side, wherein each longitudinal ruler comprises a plurality of measurement indicia therealong;
a pair of extensions rising from the rear side of the headrest base, wherein the pair of extensions are dimensioned to receive a neck therebetween.

10. The treatment positioning headrest of claim 9, wherein a height of the rear side is greater than a height of the front side.

11. The treatment positioning headrest of claim 9, wherein the depression comprises an arcuate shape along an entire length between the front side and the rear side.

12. The treatment positioning headrest of claim 9, further comprising a plurality of apertures through the planar flange, the plurality of apertures configured to receive a fastener therethrough.

13. The treatment positioning headrest of claim 9, wherein the longitudinal rulers are etched into the pair of lateral sides.

14. The treatment positioning headrest of claim 9, further comprising reference indicia disposed on each lateral side adjacent to the rear side, wherein the reference indicia is associated with a contour of a plurality of headrest bases.

15. The treatment positioning headrest of claim 9, wherein a depth of the depression defined in the upper side is less than a height of the front side, the rear side, and the pair of lateral sides.

16. The treatment positioning headrest of claim 9, wherein the longitudinal rulers are mirrored relative to each other.

17. The treatment positioning headrest of claim 9, further comprising an arcuate cutout defined between the pair of extensions.
